(19) United States
(12) Patent Application Publication
(10) Pub. No.: US 2024/0127929 A1
(43) Pub. Date: Apr. 18, 2024

(54) SYSTEM AND METHOD FOR REVIEWING ANNOTATED MEDICAL IMAGES

(71) Applicant: Shanghai United Imaging Intelligence Co., LTD., Xuhui District (CN)

(72) Inventors: Arun Innanje, Cambridge, MA (US); Abhishek Sharma, Cambridge, MA (US); Xiao Chen, Cambridge, MA (US); Zhanhong Wei, Cambridge, MA (US); Terrence Chen, Cambridge, MA (US)

(73) Assignee: Shanghai United Imaging Intelligence Co., LTD., Xuhui District (CN)

(21) Appl. No.: 17/966,948

(22) Filed: Oct. 17, 2022

(51) Int. Cl. G16H 30/40; G06F 3/0482; G06F 3/0484; G06F 40/169; G06V 10/776; G06V 20/70

(52) U.S. Cl. CPC G16H 30/40; G06F 3/0482; G06F 3/0484; G06F 40/169; G06V 10/776; G06V 20/70; G06V 2201/03

(57) ABSTRACT

Disclosed is a method and a system for reviewing annotated medical images. The method includes receiving a dataset of medical images comprising one or more pre-existing annotations therein. The method also includes displaying, via a first graphical user interface, at a given instance, one of the medical images, and detecting a first input comprising a modification of at least one pre-existing annotation in the one of the medical images being displayed to define at least one modified annotation therefor and a reference for the at least one modified annotation to be associated therewith. The method also includes displaying, via a second graphical user interface, the one of the medical images having the at least one modified annotation and the associated reference for the at least one modified annotation, and detecting a second input comprising one of verification, correction, or rejection of the at least one modified annotation.

### TECHNICAL FIELD

The aspects of the disclosed embodiments relates generally to medical imaging, and in particular to a workflow for reviewing annotated medical images.

### BACKGROUND

Advances in cardiac imaging techniques have made it possible to obtain high resolution images of the complete cardiac cycle. Magnetic Resonance Imaging (MRI) is emerging as a powerful tool for the imaging of cardiac abnormalities. MRI has become an invaluable medical diagnostic tool because of its ability to obtain high resolution in vivo images of a selected portion of the body, without invasion or use of ionizing radiation. In such imaging, a main magnetic field is applied longitudinally to an elongated generally cylindrical measurement space. MRI allows for precise morphological characterization of heart structures. For instance, contour feature extraction from the cardiac image may be used for computational purposes such as computing a measure of the volume of the blood pool in a ventricle when the extracted contour is the inner heart wall (endocardium), and an ejection fraction may then be computed from such ventricular volume measures of the end of diastole and end of systole phase positions.

Cardiac function and strain analysis using dynamic images from MRI requires the user to annotate large number of image frames across slices between of end-diastolic (ED) and end-systolic (ES) frames on short and long axis data and also define the region of the left ventricle of the heart structures in the long axis series. The process of annotation of such medical images is extremely difficult and time-consuming. This process require “expert” users (such as, physicians including radiologists and oncologists, surgeons, etc.) with required skill set and training to be capable of making such annotations, and who may already be in short supply. To reduce or eliminate manual annotating of the medical images, AI based algorithms involving machine learning are being increasingly employed to calculate the initial results which may then be edited (corrected) by the users, if required; which significantly reduce the manual effort otherwise required.

However, as may be contemplated, such AI based algorithms would require a large set of annotated images data at the first place for its training purposes to achieve a certain desired accuracy. Now unlike generating other types of image annotation data, the limitation with task of annotating large set of medical images is that such task cannot be generally crowd-sourced because of the high skill level and training required for the users for such purpose. That being said, over a period of time the users would expect that the manually annotated data will lead to improvement in the accuracy of the algorithm. But one of the problems with this assumption is the quality of annotated data. As may be appreciated, an output of the AI based algorithms involving machine learning is heavily dependent on quality of the training data.

Now in order to determine if the medical images data is correctly annotated, a review of the annotated data is required (which is a common practice in most cases). Herein, a first user may make modification(s) in the pre-existing annotated data if he/she may find any one or more of the annotations to be incorrect, and thereafter usually a second user (or same first user) may review the modification(s) (as a second opinion) for verification and confirmation. With current technologies, the existing manually annotated data can be loaded back into a Cognitive Machine Learning (CMR) application and is reviewed by the users (herein, the said first user and the second user). That is, the existing approach involves loading the data into the workflow and manually reviewing all the frames. However, there is no visual guidance for the user to help him/her navigate only through the modified annotations. Thus, such review process may take as much time as repeating the annotation process in itself, which is a highly inefficient approach. There are some other known approaches to address this problem which compare the original contour points with the newly annotated points to generate some metrics. However, this process does not give any visual feedback on the accuracy of the annotation and also this is not integrated into the workflow which makes it difficult for supervisors to review the annotation done by a technician, for example.

In light of the foregoing discussion, there exists a need for an efficient approach for Cardiac MR workflows which will make the review process more targeted and reduce the overall review time required, making it more efficient. Further limitations and disadvantages of conventional and traditional approaches will become apparent to one of skill in the art through comparison of such systems with some aspects of the present disclosure as set forth in the remainder of the present application with reference to the drawings.

### SUMMARY

The aspects of the disclosed embodiments provide a method and a system for reviewing annotated medical images, substantially as shown in and/or described in connection with at least one of the figures, as set forth more completely in the claims.

In an example, the aspects of the disclosed embodiments provide a method for reviewing annotated medical images. In one embodiment, the method includes receiving a dataset of medical images, with each medical image in the dataset of medical images comprising one or more pre-existing annotations therein. The method also includes displaying, via a first graphical user interface, at a given instance, one of the medical images from the dataset of medical images. The method also includes detecting a first input via the first graphical user interface, the first input comprising a modification of at least one pre-existing annotation of the one or more pre-existing annotations in the one of the medical images being displayed to define at least one modified annotation therefor, the first input further comprising a reference for the at least one modified annotation to be associated therewith. The method also includes displaying, via a second graphical user interface, the one of the medical images having the at least one modified annotation and the associated reference for the at least one modified annotation. The method also includes detecting a second input via the second graphical user interface, the second input comprising one of verification, correction, or rejection of the at least one modified annotation.

In a possible implementation form, the dataset of medical images comprises a time-series of medical scans of an organ of a patient.

In a possible implementation form, the method further includes processing other of medical scans of the time-series of medical scans based on the one of the medical images, as part of the time-series of medical scans, having the at least one modified annotation and the reference for the at least one modified annotation, to determine respective correlations between the at least one modified annotation in the one of the medical images and one or more pre-existing annotations in the other of medical scans of the time-series of medical scans. The method also includes automatically modifying at least one pre-existing annotation of the one or more pre-existing annotations in each of the other of medical scans of the time-series of medical scans to define at least one automatically modified annotation therefor based on the determined respective correlations. The method also includes automatically generating a reference for the at least one automatically modified annotation for each of the other of medical scans of the time-series of medical scans, to be associated therewith.

In a possible implementation form, the method further includes displaying, via the second graphical user interface, at a given instance, one of the other of medical scans of the time-series of medical scans having the at least one automatically modified annotation and the associated automatically generated reference for the at least one automatically modified annotation. The method also includes detecting a third input via the second graphical user interface, the third input comprising one of verification, correction, or rejection of the at least one automatically modified annotation.

In a possible implementation form, the method further includes displaying, via the second graphical user interface, the one of the medical images, with the one or more pre-existing annotations therein being displayed in one color and the at least one modified annotation therein being displayed in a different color.

In a possible implementation form, the method further includes displaying, via the first graphical user interface, thumbnails for each of the medical images in the dataset of medical images. The method also includes detecting a fourth input via the first graphical user interface, the fourth input comprising selection of one of thumbnails. The method also includes displaying, via the first graphical user interface, medical image corresponding to the selected one of thumbnails at the given instance.

In a possible implementation form, the method further includes displaying, via the second graphical user interface, thumbnails for each one of the medical images having the at least one modified annotation, along with a visual indicator to indicate if the displayed thumbnail of the one of the medical images having the at least one modified annotation has the associated reference for the at least one modified annotation therewith.

In a possible implementation form, the visual indicator is in form of one or more of background highlight, tooltips, border color, text, or icons overlaid on the at least one modified annotation.

In a possible implementation form, the reference for the at least one modified annotation is in form of one or more of a text note, an audio note, or a video recording.

In a possible implementation form, the method further generating a summary report comprising a list of the at least one modified annotation and the associated reference for the at least one modified annotation for all medical images in the dataset of medical images.

In another example, the aspects of the disclosed embodiments provide a system for reviewing annotated medical images. In one embodiment, the system includes a memory configured to store a dataset of medical images, with each medical image in the dataset of medical images comprising one or more pre-existing annotations therein. The system also includes a processing arrangement configured to display, via a first graphical user interface, at a given instance, one of the medical images from the dataset of medical images; detect a first input via the first graphical user interface, the first input comprising a modification of at least one pre-existing annotation of the one or more pre-existing annotations in the one of the medical images being displayed to define at least one modified annotation therefor, the first input further comprising a reference for the at least one modified annotation to be associated therewith; display, via a second graphical user interface, the one of the medical images having the at least one modified annotation and the associated reference for the at least one modified annotation; and detecting a second input via the second graphical user interface, the second input comprising one of verification, correction, or rejection of the at least one modified annotation.

In a possible implementation form, the dataset of medical images comprises a time-series of medical scans of an organ of a patient.

In a possible implementation form, the processing arrangement is further configured to process other of medical scans of the time-series of medical scans based on the one of the medical images, as part of the time-series of medical scans, having the at least one modified annotation and the reference for the at least one modified annotation, to determine respective correlations between the at least one modified annotation in the one of the medical images and one or more pre-existing annotations in the other of medical scans of the time-series of medical scans; automatically modify at least one pre-existing annotation of the one or more pre-existing annotations in each of the other of medical scans of the time-series of medical scans to define at least one automatically modified annotation therefor based on the determined respective correlations; and automatically generate a reference for the at least one automatically modified annotation for each of the other of medical scans of the time-series of medical scans, to be associated therewith.

In a possible implementation form, the processing arrangement is further configured to display, via the second graphical user interface, at a given instance, one of the other of medical scans of the time-series of medical scans having the at least one automatically modified annotation and the associated automatically generated reference for the at least one automatically modified annotation; and detect a third input via the second graphical user interface, the third input comprising one of verification, correction, or rejection of the at least one automatically modified annotation.

In a possible implementation form, the processing arrangement is further configured to display, via the second graphical user interface, the one of the medical images, with the one or more pre-existing annotations therein being displayed in one color and the at least one modified annotation therein being displayed in a different color.

In a possible implementation form, the processing arrangement is further configured to display, via the first graphical user interface, thumbnails for each of the medical images in the dataset of medical images; detect a fourth input via the first graphical user interface, the fourth input comprising selection of one of thumbnails; and display, via the first graphical user interface, medical image corresponding to the selected one of thumbnails at the given instance.

In a possible implementation form, the processing arrangement is further configured to display, via the second graphical user interface, thumbnails for each one of the medical images having the at least one modified annotation, along with a visual indicator to indicate if the displayed thumbnail of the one of the medical images having the at least one modified annotation has the associated reference for the at least one modified annotation therewith.

In a possible implementation form, the visual indicator is in form of one or more of background highlight, tooltips, border color, text, or icons overlaid on the at least one modified annotation.

In a possible implementation form, the reference for at least one modified annotation is in form of one or more of a text note, an audio note, or a video recording.

In a possible implementation form, the processing arrangement is further configured to generate a summary report comprising a list of the at least one modified annotation and the associated reference for the at least one modified annotation for all medical images in the dataset of medical images.

It is to be appreciated that all the aforementioned implementation forms can be combined. It has to be noted that all devices, elements, circuitry, units, and means described in the present application could be implemented in the software or hardware elements or any kind of combination thereof. All steps which are performed by the various entities described in the present application as well as the functionalities described to be performed by the various entities are intended to mean that the respective entity is adapted to or configured to perform the respective steps and functionalities. Even if, in the following description of specific embodiments, a specific functionality or step to be performed by external entities is not reflected in the description of a specific detailed element of that entity that performs that specific step or functionality, it should be clear for a skilled person that these methods and functionalities can be implemented in respective software or hardware elements, or any kind of combination thereof. It will be appreciated that features of the present disclosure are susceptible to being combined in various combinations without departing from the scope of the present disclosure as defined by the appended claims.

Additional aspects, advantages, features, and objects of the present disclosure would be made apparent from the drawings and the detailed description of the illustrative implementations construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams in which:

FIG. 1 is a flowchart of a method for reviewing annotated medical images, in accordance with an embodiment of the present disclosure;

FIG. 2 is an exemplary depiction of a first graphical user interface to allow a user to make modification(s) to pre-existing annotations in the annotated medical images, in accordance with an embodiment of the present disclosure;

FIG. 3 is an exemplary depiction of a second graphical user interface to allow a user to review the modification(s) to the pre-existing annotations in the annotated medical images, in accordance with an embodiment of the present disclosure; and

FIG. 4 is a block diagram of a system for reviewing annotated medical images, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

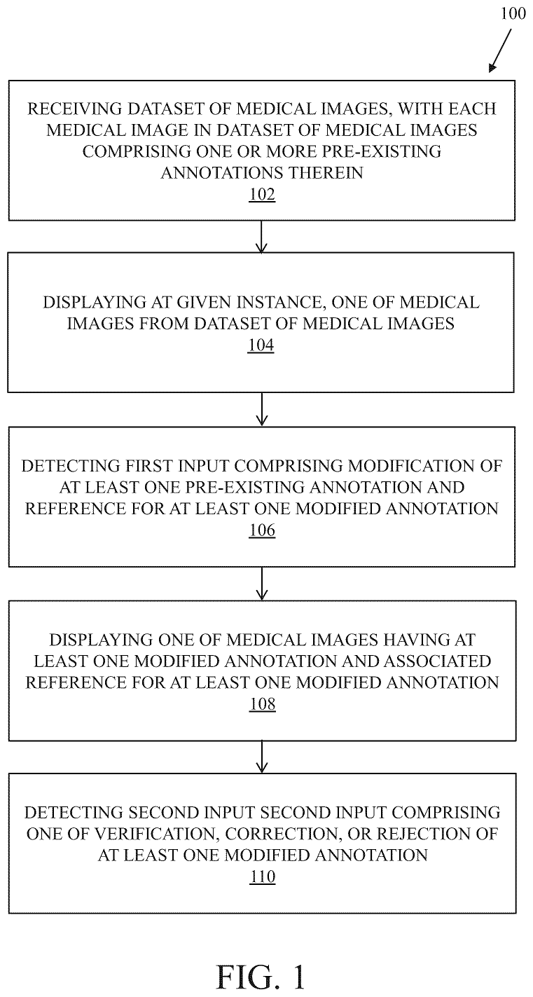

The exemplary embodiments are related to methods and systems for reviewing annotated medical images. As is shown in FIG. 1, one embodiment of the method for reviewing annotated medical images includes receiving 102 a dataset of medical images, with each medical image in the dataset of medical images comprising one or more pre-existing annotations therein. One of the medical images from the dataset of medical images is displayed 104 via a first graphical user interface. A first input via the first graphical user interface is detected 106. The first input comprises a modification of at least one pre-existing annotation of the one or more pre-existing annotations in the one of the medical images being displayed to define at least one modified annotation therefor. The first input further comprises a reference for the at least one modified annotation to be associated therewith.

One of the medical images having the at least one modified annotation and the associated reference for the at least one modified annotation is displayed 108 via a second graphical user interface. A second input is detected 110 via the second graphical user interface. The second input includes one of verification, correction, or rejection of the at least one modified annotation.

The aspects of the disclosed embodiments provide a natural way of analyzing the medical images with pre-existing annotations and making modifications to the pre-existing annotations by integrating the review mechanism into the existing workflow. The present embodiments introduce a forensics mode in the medical imaging workflow which allows a user to run a forensics analysis of the actions performed on the workflow so far. This is different from a typical undo/redo stack provided by known medical imaging workflow. In the sense, the present embodiments introduce markers/tags for the modified annotations of the workflow that allows users to get a birds eye view of the actions performed on the workflow so far and quickly review and reannotate the parts of the workflows that adds the most value for their time and use case. This approach is particularly meaningful to implement for cardiac imaging workflow as dynamic cardiac imaging workflows provides mechanism to view the detected contour in each frame of across slices, but could be applied for review of annotated data for any kind of medical images without any limitations.

Referring to FIG. 1, illustrated is a flowchart of a method 100 for reviewing annotated medical images, in accordance with an embodiment of the present disclosure. As used herein, the medical images include image data generated by medical scanning of an organ of a patient, such as ultrasonic data, magnetic resonance imaging (MRI) data, mammography data, and the like. Such medical images are stored in a standard image format such as the Digital Imaging and Communications in Medicine (DICOM) format, and in a memory or a computer storage system such as a Picture Archiving and Communication System (PACS), a Radiology Information System (RIS), and the like. Further, these medical images can be retrieved from storage or received directly from an imaging source such as an MR scanner, CT scanner, PET scanner, and the like. The medical images from multiple modalities are processed and analyzed to extract the quantitative and qualitative information. Quantitative information can include kinetics information and biochemical information. Kinetics features can be extracted from a time sequence of image data, such as MRI image data. Biochemical information can be extracted from a spectroscopic analysis of MRS data. Morphological features can be extracted from MRI images, ultrasound images, x-ray images, or images of other modalities.

At step 102, the method 100 includes receiving a dataset of medical images, with each medical image in the dataset of medical images comprising one or more pre-existing annotations therein. Generally, annotations are related to an image or slice. Image annotating, in a broad sense, includes any technique which allows a user to label, point to or otherwise indicate some feature of the image that is the focus of attention, including textual commentary. Providing an individual with the ability to add symbols, labels and captions to describe the contents of an image or to convey a concept and direct the viewer to important features of an image has been established. It has been long accepted that assigning captions or a definition and providing an option to write a legend that further describes a region of interest that is unique to an image allows the user to convey intellectual information regarding the structures in the image itself.

As used herein, annotations can include multi-media formats such as text, graphics, voice, etc. Annotations can be displayed as representations such as geometric objects, freehand drawings, measurement lines, text boxes, etc., overlaying the image slices, or separate from the associated image such as a sidebar, palate, icons, etc. The annotations can be visual and/or audible. The annotations including the size, location, orientation, etc. are stored with the associated image. The annotations and associated image can be stored as pieces of an object, package, etc., or separately and dynamically linked via a database or image meta-data. For the purpose of the present disclosure, the annotations has been described in terms of marking on the images which may trace a finding, like the most well defined border to the lesion. Generally, an annotation will include one or more of the following: a region of interest, a pointer, and textual information such as a symbol, a label and/or a caption. The visible portion of the annotation on the image may include the region of interest, the pointer and the symbol. The region of interest, pointer and symbol may allow the user, for example, to identify anatomical structures that convey relevant information about that image.

In particular, the region of interest is the visible portion of the annotation that is of interest. For example, in the medical field, a region of interest could be a feature or structure on an image (e.g., pathology, tumor, nerve) that conveys a clinical or research finding. While any manner to mark the region of interest will suffice, a user generally draws a point, line, or polygon to indicate a region of interest. The region of interest may be described by a set of points that may define a polygon, polyline or set of points, for example. A polygon may be used when the region of interest is a well-defined area, the polyline (or edge) may be used when the separation of regions is of interest and the points may be used when the interesting features are too small to practically enclose with a polygon. The pointer for the annotation is partially defined by the user and partially computed based on where the user initially places it. For example, the user selects where the tail of the pointer should appear, and an algorithm calculates the closest point on the region of interest to place the pointer tip. The textual information that is defined by the annotation methodology and includes the symbol, label and caption. Providing the ability to add textual information about the annotation enables the user to comment or add their expert knowledge on contents of an image in the form of a symbol, label and caption. The comments may refer to a detail of the image or the annotated image as a whole.

In an example, a specialist, such as a radiologist or healthcare practitioner, may analyze the medical images (and/or image slices) to add the said pre-existing annotations therein. For instance, cardiac function and strain analysis using dynamic images from MRI requires the specialist to review large number of image frames across slices between of end-diastolic (ED) and end-systolic (ES) frames on short and long axis data and also define the region of the left ventricle of the heart structures in the long axis series. A patient study can include a series of parallel slices spanning a region of the patient, e.g., 20-50 cm or more. The thickness of the slice varies with the imaging modality and is typically 1-5 mm. During the review process, the specialist may make annotations on at least some of the images and includes details in the annotations such as lesion measurements. When annotating images, the goal of the specialist is to annotate an image slice with a best example of a finding. For example, in a lesion, the best example can include an image slice with the most well defined border to the lesion, or an image slice which shows a maximum dimension of the lesion. In other examples, an artificial intelligence based algorithm may analyze the medical images (and/or image slices) to add the said pre-existing annotations therein, without any limitations.

In some examples, the medical images with the pre-existing annotations may also include accompanying information, including patient information regarding a patient such as a patient name of a patient to be radiographed (image-generated), patient ID, age, sex and the like; examination information such as examination date, examination ID, part information, radiographing (image-generating) condition (body position, radiographing (image-generating) direction and the like), image recording modality information and the like; image data information such as pixel number of a medical image, bit number, designated output size, reading pixel size, maximum density and the like; etc.

As discussed, in order to determine if the medical images data is correctly annotated, a review of the annotated data is required (which is a common practice in most cases). Herein, a first user may make modification(s) in the pre-existing annotated data if he/she may find any one or more of the annotations to be incorrect, and thereafter usually a second user (or same first user) may review the modification(s) (as a second opinion) for verification and confirmation. The method 100 provides a natural way of analyzing the medical images with pre-existing annotations and making modifications to the pre-existing annotations by integrating the review mechanism into the existing workflow, as discussed in the proceeding paragraphs in detail.

In an embodiment, the dataset of medical images may include a time-series of medical scans of an organ of a patient. For instance, for heart as the organ, cardiac function and flow represent dynamic processes that are affected by a variety of physiologic influences such as respiration, blood pressure, heart rate, exercise, or medication. The underlying myocardial and valvular movements are characterized by only a limited degree of periodicity which is often further compromised in patients with cardiovascular disease. Clinical evaluations of the heart often require a comprehensive three-dimensional coverage of the myocardium. This may efficiently be accomplished using real-time MRI by sequential “multi-slice movie” acquisitions followed by the application of advanced post-processing software. For example, the acquisition part may involve 12 directly neighboring sections each with movies of 10 s duration, so that the anatomical/functional exam of the entire heart will be completed within 2 min. Such real-time MRI allows for direct monitoring of both ventricles during ergometry in patients with ischemic or other cardiomyopathies or during stress tests in young children with congenital heart defects before and after repair. Other promising applications will be restrictive and constrictive cardiomyopathies where the ability to detect direct interactions between the ventricles adds important information. Such studies may even help in the early diagnosis of pathologies such as diastolic dysfunction. With respect to blood flow, real-time flow analyses provide information about beat-to-beat variations in flow velocity and volume simultaneously in more than one vessel. Future extensions will be MRI-guided catheterization and interventions that depend on real-time imaging with catheter tracking.

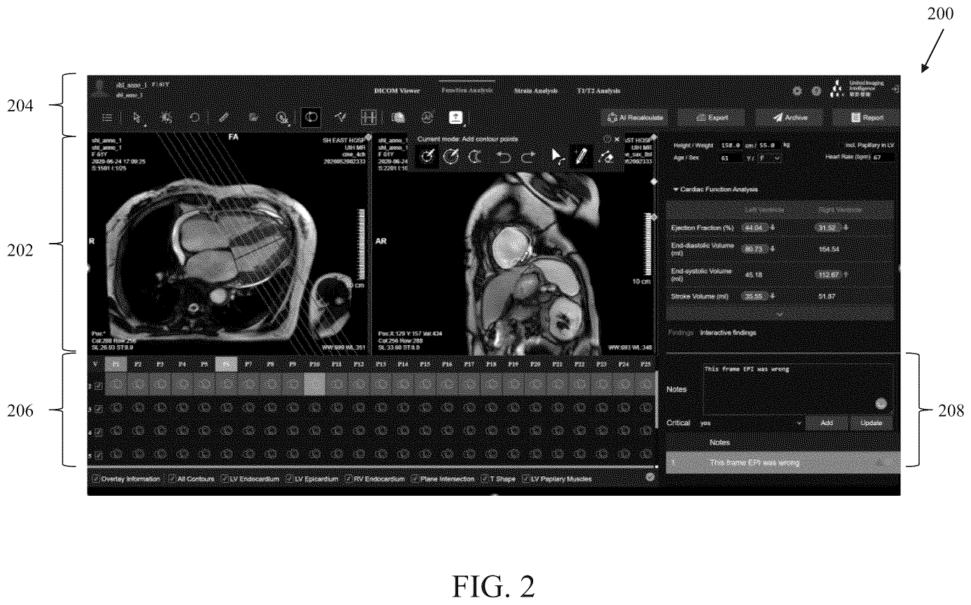

At step 104, the method 100 includes displaying, via a first graphical user interface, at a given instance, one of the medical images from the dataset of medical images. FIG. 2 illustrates a depiction of the first graphical user interface (as represented by reference numeral 200), in accordance with an embodiment of the present disclosure. The first graphical user interface 200 may include an image display section 202, which provides one or more image windows (in the illustrated example, two image windows) to display the medical image(s) being analyzed at the given instance. In one or more examples, the image display section 202, with two image windows, may display two different views of a same medical image being analyzed by the user at the given instance. The first graphical user interface 200 may further include a tool palette 204 which includes a set of tool (such as, but not limited to, draw tool, erase tool, etc., which may be contemplated by a person skilled in the art) for the user to make annotations and/or make modifications to the pre-existing annotations in the displayed medical image(s) in the image display section 202. For example, the tool palette 204 may include a plurality of selectable control tools for use in segmenting and/or editing the cross-sectional images, including a contouring/segmenting tool for defining contours and/or regions of an anatomical feature in the medical image. The first graphical user interface 200 may further include a grid section 206 which may display thumbnails for each of the medical images in the dataset of medical images (or at least the number of thumbnails of medical images that could be displayed on a display device at a given instance). The first graphical user interface 200 may further include a notes section 208 which may allow the user to add notes (such as, text notes) related to the made annotations and/or the made modifications to the pre-existing annotations (as discussed hereinafter).

At the step 104 of the method 100, the first graphical user interface 200 may display the medical image corresponding to the selected thumbnail from the grid section 206, and may display that medical image in the image display section 202. As discussed, the dataset of medical images may include a time-series of medical scans of an organ of a patient, such as the heart for analyzing cardiac functions. In such case, the plurality of images, as generated during the imaging process, may all be displayed in the grid section 206 of the first graphical user interface 200, which may display thumbnails for each of the medical images in the dataset of medical images (or at least the number of thumbnails of medical images that could be displayed on a display device implementing the first graphical user interface 200 at the given instance). In the present embodiments, the first graphical user interface 200 may be configured to receive an input, namely a fourth input, in which the fourth input may include selection of one of thumbnails as being displayed in the grid section 206 of the first graphical user interface 200. In case of receipt of such input, the first graphical user interface 200 may display the medical image corresponding to the selected one of thumbnails at the image display section 202 to be analyzed by the user at the given instance.

At step 106, the method 100 includes detecting an input, namely a first input, via the first graphical user interface 200. In the present embodiments, the first input includes a modification of at least one pre-existing annotation of the one or more pre-existing annotations in the one of the medical images being displayed to define at least one modified annotation therefor. As the selected medical image is being displayed at the image display section 202 to be analyzed by the user at the given instance, the user may review the pre-existing annotations therein. In case, the user may find one or more of the pre-existing annotations being incorrect, the user may have the option to correct the same using the first graphical user interface 200. For this purpose, the user may select one or more of available tools, such as the draw tool or the erase tool, from the tool palette 204, and make the required corrections in the pre-existing annotation. Such modified/corrected form of the pre-existing annotation has been referred to as “modified annotation” for the purposes of the present disclosure. In the present embodiments, the first input may also include a reference for the at least one modified annotation to be associated therewith. In one or more embodiments, the reference for the at least one modified annotation is in form of one or more of a text note, an audio note, or a video recording. For example, the user may add the reference for the modified annotation, such as a text note, by typing into the notes section 208 of the first graphical user interface 200 a comment which is related to the made annotations and/or the made modifications to the pre-existing annotations. The said other types of references including the audio note or the video recording may alternatively or additionally be added, as may be contemplated by a person skilled in the art without departing from the spirit and the scope of the present disclosure.

As discussed, for reviewing annotations in the medical images, a first user may initially make modification(s) in the pre-existing annotated data if he/she may find any one or more of the annotations to be incorrect (as discussed in the preceding paragraphs), which is achieved by means of the first graphical user interface 200 as described therein. Thereafter, it may usually be required that a second user (or same first user) may review the modification(s) (as a second opinion) for verification, correction, or rejection. In such case, it may be desired to provide another graphical user interface which may be “custom designed” for the workflow related to review of the modifications made in the pre-existing annotations in order to make the process more efficient, rather than using the same graphical user interface (i.e., the first graphical user interface 200) for the said purpose. The proceeding paragraphs describes details for such a graphical user interface for a second user (which, in some cases, could be same as the first user) along with the workflow for the second user to achieve the said purpose. It may be appreciated that, herein, if the two said users are the same person, then that person may switch from the first graphical user interface 200 to the said other graphical user interface on a same device or the like, using an option provided in application software (as may be contemplated). On the other hand, in case of the second user being different and possibly working on a different device, then an output of the workflow from the first graphical user interface 200 may be exported to be imported (or loaded) into the other graphical user interface. Such details will become clearer with reference to discussion of system aspect of the present disclosure later in the description.

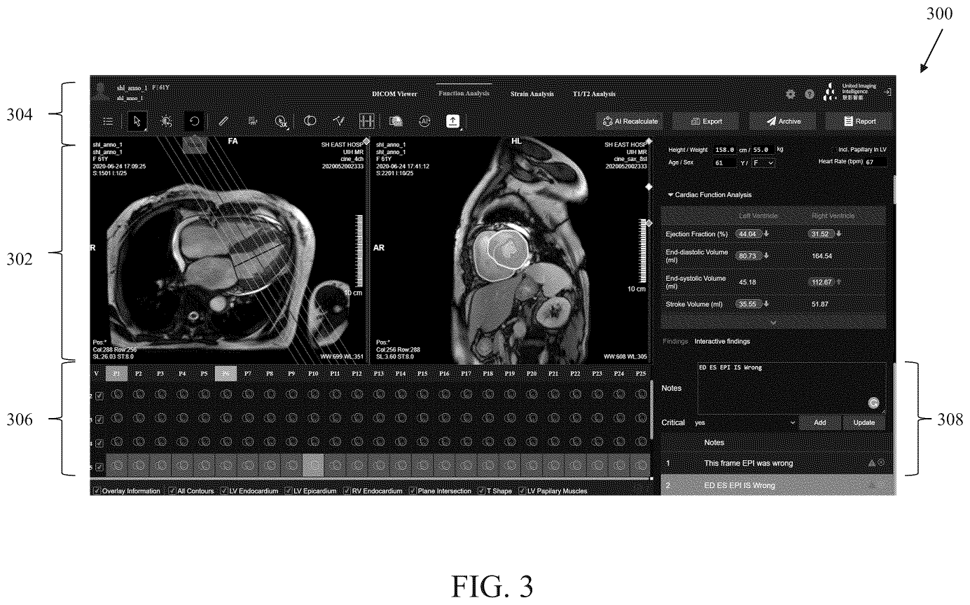

At step 108, the method 100 includes displaying, via a second graphical user interface, the one of the medical images having the at least one modified annotation and the associated reference for the at least one modified annotation. FIG. 3 illustrates a depiction of the second graphical user interface (as represented by reference numeral 300), in accordance with an embodiment of the present disclosure. Similar to the first graphical user interface 200, the second graphical user interface 300 may also include an image display section 302, which provides one or more image windows (in the illustrated example, two image windows) to display the medical image(s) being analyzed at the given instance. Again herein, the image display section 302, with two image windows, may display two different views of a same medical image being analyzed by the user at the given instance. Also, similar to the first graphical user interface 200, the second graphical user interface 300 may include a tool palette 304 which includes a set of tool (such as, but not limited to, draw tool, erase tool, etc., which may be contemplated by a person skilled in the art) for the user to make annotations and/or make modifications to the pre-existing annotations in the displayed medical image(s) in the image display section 302. Further similar to the first graphical user interface 200, the second graphical user interface 300 may include a grid section 306. Herein, the grid section 306 of the second graphical user interface 300 may display thumbnails for each of the medical images with modified annotations therein (or at least the number of thumbnails of medical images with modified annotations that could be displayed on a display device implementing the second graphical user interface 300, at the given instance). Furthermore similar to the first graphical user interface 200, the second graphical user interface 300 may include a notes section 308 which may allow the user to add notes (such as, text notes) related to the made annotations and/or the made modifications to the modified annotations (as discussed hereinafter).

At the step 108 of the method 100, the second graphical user interface 300 may display the medical image corresponding to the selected thumbnail from the grid section 306, and may display that medical image in the image display section 302. Herein, the plurality of images with the modified annotations may all be displayed in the grid section 306 of the second graphical user interface 300, which may display thumbnails for each of such medical images with the modified annotations (or at least the number of thumbnails of medical images that could be displayed on the display device implementing the second graphical user interface 300 at the given instance). Herein, the second graphical user interface 300 may be configured to receive an input, similar to the fourth input (as discussed above), in which the said input may include selection of one of thumbnails as being displayed in the grid section 306 of the second graphical user interface 300. In case of receipt of such input, the second graphical user interface 300 may display the medical image corresponding to the selected one of thumbnails at the image display section 302 to be analyzed by the user at the given instance.

In an embodiment, the method 100 includes displaying, via the second graphical user interface 300, thumbnails for each one of the medical images having the at least one modified annotation, along with a visual indicator to indicate if the displayed thumbnail of the one of the medical images having the at least one modified annotation has the associated reference for the at least one modified annotation therewith. That is, the second graphical user interface 300 may be configured such that all the images with the modified annotations may being displayed in the grid section 306 having the corresponding reference associated therewith may be highlighted by use of the said visual indicator for easy reference of the user using the second graphical user interface 300. In the present embodiments, the visual indicator is in form of one or more of background highlight, tooltips, border color, text, or icons overlaid on the at least one modified annotation. For example, the thumbnails of images having the corresponding reference associated therewith, as being displayed in the grid section 306, may be highlighted with a colored border for easy visual reference of the user, so as to enable the user to only select such thumbnails for the corresponding medical image to be displayed for further analysis.

In an embodiment, the method 100 includes displaying, via the second graphical user interface 300, the one of the medical images, with the one or more pre-existing annotations therein being displayed in one color and the at least one modified annotation therein being displayed in a different color. That is, the second graphical user interface 300 may display the selected medical showing the pre-existing annotation(s) therein in one color and the modified annotation(s) therein in another color. Herein, in particular, the selected image being displayed in the image display section 302 of the second graphical user interface 300 may be used to display the selected medical showing the pre-existing annotation(s) therein in one color (say yellow color) and the modified annotation(s) therein in another color. This is done for the user using the second graphical user interface 300 to be able to easily distinguish between the pre-existing annotation(s) and the modified annotation(s) therein in the medical image being displayed in the image display section 302 at the given instance.

At step 110, the method 100 includes detecting an input, namely a second input, via the second graphical user interface 300. In the present embodiments, the second input includes one of verification, correction, or rejection of the at least one modified annotation. In other words, the second input includes one of verification, correction, or rejection of the one or more pre-existing annotations in the one of the medical images. As the selected medical image is being displayed at the image display section 302 to be analyzed by the user at the given instance, the user may review the modified annotations therein. Herein, the user may also refer to the reference(s) as shown in the notes section 308 of the second graphical user interface 300, such as a text note, which is related to the made modifications to the pre-existing annotations. In case, the user may find one or more of the modified annotations being incorrect, the user may have the option to correct the same using the second graphical user interface 300. For this purpose, the user may select one or more of available tools, such as the draw tool or the erase tool, from the tool palette 304, and make the required corrections in the modified annotation. In some examples, the user may further provide an input as a reference for the made corrections in the modified annotations, for records. Similar to the reference for the modified annotation, the present reference for the correction in the modified annotation may also be in form of one or more of a text note, an audio note, or a video recording (as described).

In the embodiments of the present disclosure, with the use of the first graphical user interface 200 and the second graphical user interface 300, it is possible to double check the annotations in the medical images. For instance, in case of the pre-existing annotations in the medical images having been made by a “junior operator,” such pre-existing annotations may be reviewed by a “senior operator” and be modified (if required) using the first graphical user interface 200; and such modification may further be reviewed by the same “senior operator” or someone with even higher expertise, say a “supervisor” (like a surgeon), using the second graphical user interface 300. The implementation of the second graphical user interface 300 enables for quick selection of images with only modified annotations and clearly highlights the modified annotations therein for quick reference of the “supervisor” to perform the review, making the workflow significantly more efficient, as compared to say conventional workflows which provide no visual guidance for the user to help him/her navigate only through the modified annotations. The present disclosure provides visual indicators (markers/tags) for the modified annotations in the workflow that allows the user to get a birds eye view of the actions performed on the workflow so far, and quickly review and reannotate the parts of the workflows that adds the most value for their time and use case.

Further, as discussed, in order to reduce or eliminate manual annotating of the medical images, AI based algorithms involving machine learning are being increasingly employed. However, such AI based algorithms would require a large set of annotated images data at the first place for its training purposes to achieve a certain desired accuracy. Using teachings of the present disclosure, it may be possible to implement the AI based algorithms to make the initial set of annotations to the medical images (with their current training) and then those annotations (pre-existing annotations) be manually modified (corrected), if required, by a first user; and then further manually checked for accuracy, by a second user using the workflow as per the embodiments of the present disclosure (as described above), with all this being achieved in a significantly more efficient manner as compared to known techniques, thus saving in time, cost and resources otherwise involved in performing manual annotations using conventional techniques. This may further help to generate the required large set of training data for the said AI based algorithms, thus solving a bigger problem of manual annotation of the medical images which may otherwise be very costly and time-consuming.

As discussed, in the present examples, the dataset of medical images may include the time-series of medical scans of the organ of the patient. In some embodiments, the method 100 further includes processing other of medical scans of the time-series of medical scans based on the one of the medical images, as part of the time-series of medical scans, having the at least one modified annotation and the reference for the at least one modified annotation, to determine respective correlations between the at least one modified annotation in the one of the medical images and one or more pre-existing annotations in the other of medical scans of the time-series of medical scans. Herein, first, the one or more annotations for anatomical features identified in the medical images of the patient's organ may be determined; and then a dependency or hierarchy between at least two of the one or more annotations for anatomical features identified in the other medical images as part of the time-series of medical scans of the patient's organ may be determined. The method 100 further includes automatically modifying at least one pre-existing annotation of the one or more pre-existing annotations in each of the other of medical scans of the time-series of medical scans to define at least one automatically modified annotation therefor based on the determined respective correlations. That is, based on the dependency or hierarchy, the method 100 configures the AI based module to add one or more annotations for anatomical features identified in the said other medical images of the patient's organ. The method 100 may further include automatically generating a reference for the at least one automatically modified annotation for each of the other of medical scans of the time-series of medical scans, to be associated therewith. That is, based on the dependency or hierarchy, the method 100 configures the AI based module to generate comments for the added one or more annotations for anatomical features identified in the said other medical images of the patient's organ. This helps to generate a large set of annotated images data with the initial set of modified annotations, thereby reducing the manual work required for a first user (like “junior operator,” as discussed above).

The method 100 may further include displaying, via the second graphical user interface 300, at a given instance, one of the other of medical scans of the time-series of medical scans having the at least one automatically modified annotation and the associated automatically generated reference for the at least one automatically modified annotation. That is, the second graphical user interface 300 may display the medical images with the automatically modified annotation and the associated automatically generated reference, for review by the second user (such as, “supervisor” as discussed above). The method 100 may further include detecting an input, namely a third input, via the second graphical user interface 300. Herein, the third input may include one of verification, correction, or rejection of the at least one automatically modified annotation. That is, the second user may then quickly review the automatically modified annotations, thus saving in time, cost and resources otherwise involved in performing manual annotations using conventional techniques.

In an embodiment, the method 100 further includes generating a summary report comprising a list of the at least one modified annotation and the associated reference for the at least one modified annotation for all medical images in the dataset of medical images. Herein, the workflow, as described in the proceeding paragraphs, continuously monitors and maintains a state of the modifications made that may be then used for generation of the summary report. This provides a mechanism to understand the most commonly modified annotations (components) in a set of the medical images and thus helping with improving the workflow.

It should be noted that the flowchart and block diagrams in the figures illustrate the architecture, functionality, and operation of possible implementations of systems, apparatuses, methods and computer program products according to various embodiments of the invention. In this regard, each block in the flowchart or block diagrams may represent a module, segment, or portion of code, which comprises at least one executable instruction for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

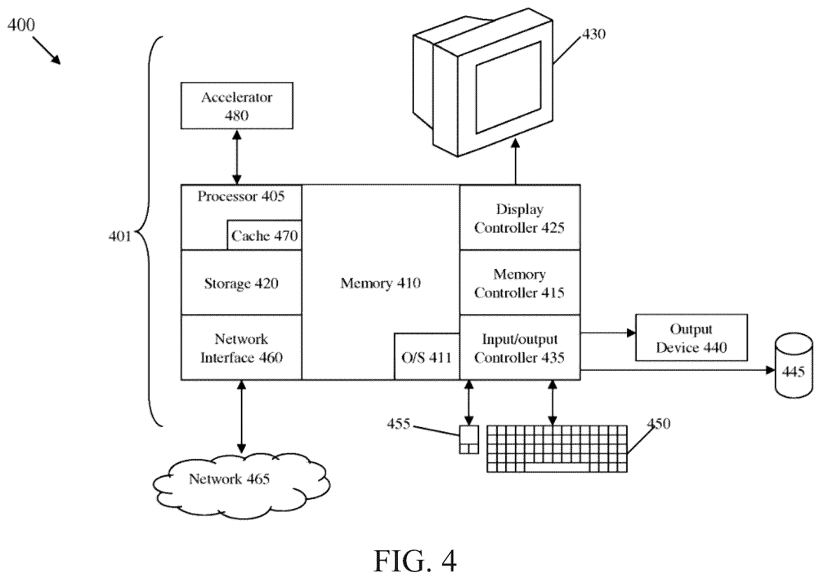

Referring to FIG. 4, illustrated is a system 400 for reviewing annotated medical images. Various embodiments and variants disclosed in reference to the method 100 above apply mutatis mutandis to the system 400. The method 100 described herein can be implemented in hardware software (e.g., firmware), or a combination thereof. In an exemplary embodiment, the method 100 described herein can be implemented in hardware, and is part of the microprocessor of a special or general-purpose digital computer, such as a personal computer, workstation, minicomputer, or mainframe computer.

In an exemplary embodiment, in terms of hardware architecture, as shown in FIG. 4, the system 400 therefore includes a general-purpose computer 401. Herein, the computer 401 includes a processing arrangement 405, a memory 440 coupled via a memory controller 445, a storage device 420, and one or more input and/or output (I/O) devices 440, 445 (or peripherals) that are communicatively coupled via a local input/output controller 435. The input/output controller 435 can be, for example, but not limited to, one or more buses or other wired or wireless connections, as is known in the art. The input/output controller 435 may have additional elements, which are omitted for simplicity, such as controllers, buffers (caches), drivers, repeaters, and receivers, to enable communications. Further, the local interface may include address, control, and/or data connections to enable appropriate communications among the aforementioned components. The storage device 420 may include one or more hard disk drives (HDDs), solid state drives (SSDs), or any other suitable form of storage.

The processing arrangement 405 is a computing device for executing hardware instructions or software, particularly that stored in memory 440. The processing arrangement 405 can be any custom made or commercially available processor, a central processing unit (CPU), an auxiliary processor among several processors associated with the computer 401, a semiconductor based microprocessor (in the form of a microchip or chip set), a macro-processor, or generally any device for executing instructions. The processing arrangement 405 may include a cache 470, which may be organized as a hierarchy of more cache levels (L1, L2, etc.). In the present examples, the processing arrangement 405 may be distributed to execute the first graphical user interface 200 and the second graphical user interface 200 in different computing devices as may be required, for instance, when the said first user of the first graphical user interface 200 and the said second user of the second graphical user interface 200 may be working out of different computing devices. Such architecture may be contemplated by a person skilled in the art and thus has not been described further for the brevity of the present disclosure.

The memory 440 can include any one or combination of volatile memory elements (e.g., random access memory (RAM, such as DRAM, SRAM, SDRAM, etc.)) and nonvolatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 440 may incorporate electronic, magnetic, optical, and/or other types of storage media. Note that the memory 440 can have a distributed architecture, where various components are situated remote from one another, but can be accessed by the processing arrangement 405.

The instructions in memory 440 may include one or more separate programs, each of which comprises an ordered listing of executable instructions for implementing logical functions. In the example of FIG. 4, the instructions in the memory 440 include a suitable operating system (OS) 411. The operating system 411 essentially controls the execution of other computer programs and provides scheduling, input-output control, file and data management, memory management, and communication control and related services.

In an exemplary embodiment, a conventional keyboard 450 and mouse 455 can be coupled to the input/output controller 435. Other output devices such as the I/O devices 440, 445 may include input devices, for example but not limited to a printer, a scanner, microphone, and the like. Finally, the I/O devices 440, 445 may further include devices that communicate both inputs and outputs, for instance but not limited to, a network interface card (NIC) or modulator/demodulator (for accessing other files, devices, systems, or a network), a radio frequency (RF) or other transceiver, a telephonic interface, a bridge, a router, and the like. The system 400 can further include a display controller 425 coupled to a display 430. In an exemplary embodiment, the system 400 can further include a network interface 460 for coupling to a network 465. The network 465 can be an IP-based network for communication between the computer 401 and any external server, client and the like via a broadband connection. The network 465 transmits and receives data between the computer 401 and external systems. In an exemplary embodiment, network 465 can be a managed IP network administered by a service provider. The network 465 may be implemented in a wireless fashion, e.g., using wireless protocols and technologies, such as Wi-Fi, WiMax, etc. The network 465 can also be a packet-switched network such as a local area network, wide area network, metropolitan area network, Internet network, or other similar type of network environment. The network 465 may be a fixed wireless network, a wireless local area network (LAN), a wireless wide area network (WAN) a personal area network (PAN), a virtual private network (VPN), intranet or other suitable network system and includes equipment for receiving and transmitting signals.

If the computer 401 is a PC, workstation, intelligent device or the like, the instructions in the memory 440 may further include a basic input output system (BIOS) (omitted for simplicity). The BIOS is a set of essential routines that initialize and test hardware at startup, start the OS 411, and support the transfer of data among the storage devices. The BIOS is stored in ROM so that the BIOS can be executed when the computer 401 is activated.

When the computer 401 is in operation, the processing arrangement 405 is configured to execute instructions stored within the memory 440, to communicate data to and from the memory 440, and to generally control operations of the computer 401 pursuant to the instructions. In exemplary embodiments, the system 400 includes one or more accelerators 480 that are configured to communicate with the processing arrangement 405. The accelerator 480 may be a field programmable gate array (FPGA) or other suitable device that is configured to perform specific processing tasks. In exemplary embodiments, the system 400 may be configured to offload certain processing tasks to an accelerator 480 because the accelerator 480 can perform the processing tasks more efficiently than the processing arrangement 405.

In the system 400, the memory 440 is configured to store the dataset of medical images, with each medical image in the dataset of medical images comprising one or more pre-existing annotations therein. Further, the processing arrangement 405 is configured to display, via the first graphical user interface 200, at the display 430, at a given instance, one of the medical images from the dataset of medical images; detect a first input via the first graphical user interface 200, the first input comprising a modification of at least one pre-existing annotation of the one or more pre-existing annotations in the one of the medical images being displayed to define at least one modified annotation therefor, the first input further comprising a reference for the at least one modified annotation to be associated therewith; display, via the second graphical user interface 300, at the display 430, the one of the medical images having the at least one modified annotation and the associated reference for the at least one modified annotation; and detecting a second input via the second graphical user interface 300, the second input comprising one of verification, correction, or rejection of the at least one modified annotation.

In one or more embodiments, the dataset of medical images comprises a time-series of medical scans of an organ of a patient. In such embodiments, the processing arrangement 405 is further configured to process other of medical scans of the time-series of medical scans based on the one of the medical images, as part of the time-series of medical scans, having the at least one modified annotation and the reference for the at least one modified annotation, to determine respective correlations between the at least one modified annotation in the one of the medical images and one or more pre-existing annotations in the other of medical scans of the time-series of medical scans; automatically modify at least one pre-existing annotation of the one or more pre-existing annotations in each of the other of medical scans of the time-series of medical scans to define at least one automatically modified annotation therefor based on the determined respective correlations; and automatically generate a reference for the at least one automatically modified annotation for each of the other of medical scans of the time-series of medical scans, to be associated therewith.

In such embodiments, the processing arrangement 405 is further configured to display, via the second graphical user interface 300, at the display 430, at a given instance, one of the other of medical scans of the time-series of medical scans having the at least one automatically modified annotation and the associated automatically generated reference for the at least one automatically modified annotation; and detect a third input via the second graphical user interface 300, the third input comprising one of verification, correction, or rejection of the at least one automatically modified annotation.

In one or more embodiments, the processing arrangement 405 is further configured to display, via the second graphical user interface 300, at the display 430, the one of the medical images, with the one or more pre-existing annotations therein being displayed in one color and the at least one modified annotation therein being displayed in a different color.

In one or more embodiments, the processing arrangement 405 is further configured to display, via the first graphical user interface 200, at the display 430, thumbnails for each of the medical images in the dataset of medical images; detect a fourth input via the first graphical user interface 200, the fourth input comprising selection of one of thumbnails; and display, via the first graphical user interface 200, at the display 430, medical image corresponding to the selected one of thumbnails at the given instance.

In one or more embodiments, the processing arrangement 405 is further configured to display, via the second graphical user interface 300, at the display 430, thumbnails for each one of the medical images having the at least one modified annotation, along with a visual indicator to indicate if the displayed thumbnail of the one of the medical images having the at least one modified annotation has the associated reference for the at least one modified annotation therewith.

In one or more embodiments, the visual indicator is in form of one or more of background highlight, tooltips, border color, text, or icons overlaid on the at least one modified annotation.

In one or more embodiments, the reference for at least one modified annotation is in form of one or more of a text note, an audio note, or a video recording.

In one or more embodiments, the processing arrangement 405 is further configured to generate a summary report comprising a list of the at least one modified annotation and the associated reference for the at least one modified annotation for all medical images in the dataset of medical images.

Thus, the method 100 and the system 400 of the present disclosure propose a novel workflow which introduces a mode in the cardiac imaging workflow for forensic analysis of modifications done to the data as part of the workflow, wherein these forensics are provided as visual indicators on the modified components of the workflow allowing users to easily identify and review the modifications done to the workflow. The proposed workflow allows users to seamlessly switch between an analysis mode involving making modifications to the annotations (as required) and a forensics mode involving review of the made modifications. Herein, the forensics mode in the medical imaging workflow of cardiac imaging allows a user to run a forensics analysis of the actions performed on the workflow so far. This is different from a typical undo/redo stack provided by most imaging applications. In a sense, the proposed workflow introduces markers/tags for the modified parts that allows users to get a birds eye view of the actions performed on the workflow so far and quickly review and reannotate the parts of the workflows that adds the most value for time and use case of the user(s) involved. The described mechanism provides a natural way of reviewing the pre-existing annotations and modifications to the pre-existing annotations in the medial images data by integrating the review mechanism into the existing workflow. This approach is particularly meaningful to implement for cardiac imaging workflow as dynamic cardiac imaging workflows provides mechanism to view the detected contour in each frame of across slices. That said, the proposed workflow may be extended to any imaging application that required forensic analysis in terms of identification and review of the modified annotations in the medical images.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable combination or as suitable in any other described embodiment of the disclosure.

1. A method for reviewing annotated medical images, the method comprising:
receiving a dataset of medical images with pre-existing annotations, the pre-existing annotations formed using an AI based algorithm, the pre-existing annotations comprising one or more of a two-dimensional (2D) contour line or three-dimensional (3D) contour line;
displaying, via a first graphical user interface, at a given instance, a medical image from the dataset of medical images with pre-existing annotations and receiving an input to modify a pre-existing annotation on the displayed medical image, the modification of the pre-existing annotation defining a modified annotation that includes a reference associated with the modified annotation;
displaying, via a second graphical user interface, a medical image with a modified annotation and an associated reference; and
detecting a second input via the second graphical user interface, the second input comprising one of a verification, a correction, or a rejection of the modified annotation.

2. The method according to claim 1, wherein the dataset of medical images with pre-existing annotations comprises a time-series of medical scans of an organ of a patient.

3. The method according to claim 2 further comprising:
processing other medical scans of the time-series of medical scans based on one of the medical images having the modified annotation and the reference for the modified annotation, to determine respective correlations between the modified annotation and one or more pre-existing annotations in the other of medical scans of the time-series of medical scans;
automatically modifying at least one pre-existing annotation of the one or more pre-existing annotations in the other medical scans of the time-series of medical scans to define at least one automatically modified annotation therefor based on the determined respective correlations; and
automatically generating a reference for the at least one automatically modified annotation to be associated therewith.

4. The method according to claim 3 further comprising:
displaying, via the second graphical user interface, at a given instance, one of the medical scans of the time-series of medical scans having the at least one automatically modified annotation and the associated automatically generated reference; and
detecting a third input via the second graphical user interface, the third input comprising one of verification, correction, or rejection of the at least one automatically modified annotation.

5. The method according to claim 1 further comprising displaying, via the second graphical user interface, medical images with the pre-existing annotations in one color in a first frame and a corresponding modified annotation in a different color in a second frame thereby allowing for on spot comparison of the pre-existing annotations and the corresponding modified annotation.

6. The method according to claim 1 further comprising:
displaying, via the first graphical user interface, thumbnail views or icons for all modified annotations;
detecting a fourth input via the first graphical user interface, the fourth input comprising a selection of one of thumbnail views; and
displaying, via the first graphical user interface, a medical image corresponding to the selected one of thumbnail views.

7. The method according to claim 1 further comprising displaying, via the second graphical user interface, a thumbnail view of all modified annotations, along with a visual indicator to indicate if the displayed thumbnail view includes the associated reference for the modified annotation.

8. The method according to claim 7, wherein the visual indicator is in a form of one or more of a background highlight, tooltips, border color, text, or icons overlaid on the modified annotation in the thumbnail view.

9. The method according to claim 1, wherein the reference for the modified annotation is in a form of one or more of a text note, an audio note, or a video recording.

10. The method according to claim 1 further comprising generating a summary report comprising a list of all modified annotations and the associated reference for all medical images in the dataset of medical images with pre-existing annotations.

11. A system for reviewing annotated medical images, the system comprising:
a memory configured to store a dataset of medical images, with each medical image in the dataset of medical images comprising one or more pre-existing annotations made to medical images in the dataset using an AI based algorithm, the one or more pre-existing annotations comprising one or more of a two-dimensional (2D) contour line or three-dimensional (3D) contour line; and
a processing arrangement configured to:
display, via a first graphical user interface, at a given instance, a medical image from the dataset of medical images with one or more pre-exiting annotations;
detect a modification of at least one of the one or more pre-existing annotations of the displayed medical image, the modification defining a modified annotation;
display, via a second graphical user interface, a medical image with a modified annotation and a reference associated with the modified annotation; and
detecting a second input via the second graphical user interface, the second input comprising one of a verification, a correction, or a rejection of the modified annotation.

12. The system according to claim 11, wherein the dataset of annotated medical images comprises a time-series of medical scans of an organ of a patient.

13. The system according to claim 12, wherein the processing arrangement is further configured to:
process other medical scans of the time-series of medical scans based on one of the medical images having the modified annotation and the reference for the modified annotation, to determine respective correlations between the modified annotation and one or more pre-existing annotations in the other of medical scans of the time-series of medical scans;
automatically modify at least one pre-existing annotation of the one or more pre-existing annotations in each of the other of medical scans of the time-series of medical scans to define at least one automatically modified annotation therefor based on the determined respective correlations; and
automatically generate a reference for the at least one automatically modified annotation to be associated therewith.

14. The system according to claim 13, wherein the processing arrangement is further configured to:
display, via the second graphical user interface, at a given instance, one of the medical scans of the time-series of medical scans having the at least one automatically modified annotation and the associated automatically generated reference; and
detect a third input via the second graphical user interface, the third input comprising one of verification, correction, or rejection of the at least one automatically modified annotation.

15. The system according to claim 11, wherein the processing arrangement is further configured to display, via the second graphical user interface medical images with the one or more pre-existing annotations in one color in a first frame and the modified annotation displayed in a different color in a second frame.

16. The system according to claim 11, wherein the processing arrangement is further configured to:
display, via the first graphical user interface, a thumbnail view of all modified annotations;
detect a fourth input via the first graphical user interface, the fourth input comprising a selection of one of the thumbnail views; and
display, via the first graphical user interface, a medical image corresponding to the selected one of thumbnail views.

17. The system according to claim 11, wherein the processing arrangement is further configured to display, via the second graphical user interface, a thumbnail view of the modified annotation, along with a visual indicator to indicate if the displayed thumbnail includes the associated reference.

18. The system according to claim 17, wherein the visual indicator is in a form of one or more of background highlight, tooltips, border color, text, or icons overlaid on the at least one modified annotation.

19. The system according to claim 11, wherein the reference for the modified annotation is in a form of one or more of a text note, an audio note, or a video recording.

20. The system according to claim 11, wherein the processing arrangement is further configured to generate a summary report comprising a list of all modified annotations and the associated reference for all medical images in the dataset of annotated medical images.
